(19) United States
(12) Patent Application Publication
(10) Pub. No.: US 2004/0187864 A1
(43) Pub. Date: Sep. 30, 2004

(54) Inhalation device and method

(75) Inventor: David E. Adams, Batavia, OH (US)

Correspondence Address: James A. Wilke, Foley & Lardner LLP, 777 East Wisconsin Avenue, Milwaukee, WI, 53202-5306, US

(73) Assignee: CINDET, LLC

(21) Appl. No.: 10/798,681

(22) Filed: Mar. 11, 2004

Related U.S. Application Data
(60) Provisional application No. 60/456,979, filed on Mar. 24, 2003.

(51) Int. Cl. A61M 11/00

(52) U.S. Cl. 128/200.14; 128/200.23

(57) ABSTRACT

An inhalation device and method to deliver a pre-selected dose of medication to a user. The inhalation device comprises an enclosure having an inhalation tube. The inhalation tube has an inlet end and an outlet end. A medication dispenser is coupled to the enclosure and is in communication with the inhalation tube. A sensor is mounted in the enclosure with at least a portion of the sensor extending into the inhalation tube. The sensor has a characteristic of responding in proportion to speed of gas flowing in a given direction within the inhalation tube. An electrical circuit is coupled to the sensor and medication dispenser and is configured to trigger the medication dispenser wherein a dose of medication is expelled into the inhalation tube upon receipt of a signal from the sensor at a predetermined gas flow speed in the inhalation tube.

### CROSS-REFERENCE TO RELATED PATENT APPLICATIONS

This application claims the benefit of U.S. Provisional Application No. 60/456,979, filed Mar. 24, 2003.

### BACKGROUND OF THE INVENTION

The present invention relates to a device and method for dispensing medication into the respiratory track of the user, and more particularly to an inhalation device having a sensor to monitor the velocity of gas moving through the inhalation device and triggering a release of medication into the gas stream.

For inhalation therapy to be effective, it is important for the medication to be dispersed in all or as many areas of the lung of the user as possible. For this to occur, the particle size of the medication, either in liquid or powder form, and the velocity of the gas flow, typically generally is referred to as air, carrying the medication into the lung needs to be coordinated. To ensure deep and uniform penetration the deposition of the medication in the lungs, transportation of the medication at a particular air speed is important. Large particles, for instance carried at an inadequate air speed will not be transported sufficiently to the lungs and more likely will be deposited in the nasal or oral passages of the user before reaching the lower extremities of the lungs. Likewise, if the air speed is too high or too low, an insufficient amount of medication would reach the user's lungs.

The delivery of medication based on breath actuated, metered dose inhalers (MDI) triggered by flow rates for the dispersal of the medication can be controlled by several types of devices. The following is a brief description of several such devices.

Flow sensing resistors and other such devices, for example temperature sensitive diodes and thermistors, are all affected by the temperature of the ambient air that is being drawn through the flow tube of the inhaler and ultimately into the lungs of the user. For accurate measurements, the ambient air temperature must be determined and the temperature as it moves across and through the device must be determined. A calculation of temperature difference between such temperatures must be determined. This difference is then calibrated to determine air flow. To calculate such differential, considerable amount of conditioning electronics is necessary and such calculations are subject to errors from changing environmental conditions, such as humidity.

Hot wire anemometers can be used, however they are costly, and require considerable power to operate. It is also difficult to get repeatable results when used in air flow determinations. Anything in the periphery of the inlet tube can cause turbulence, thereby varying the resulting measurement by as much as 20% and causing the medication in the inhaler to be dispersed at the wrong flow rate. The conditioning electronics typically employed with hot wire anemometer systems are comparatively complicated.

A mechanical vane system uses a rigid vane that extends into the air flow. When acted upon, mechanical linkage from the vane transmits the motion to a mechanical potentiometer, varying its resistance. Mechanical vanes have many movable parts and require a spring or some other means to return the vane to its original position. A typical problem with mechanical vanes are that at low flow rates, air flow can be difficult to accurately measure.

A bending vane, using a strain gauge, has also been employed. Such systems have several disadvantages. The bending vane device produces an equal output in either direction of air flow. Mechanical stops or electrical circuitry must be added to prevent triggering on an exhalation as opposed to the desired inhalation by a user. Also, a low voltage output of the strain gauge requires the addition of expensive electronics for a sufficient electrical output. Strain gauges and associated electronics are expensive and because of the high gain amplification required are subject to temperature drift and vibration.

Flow control orifices can also be employed in inhalation devices. By changing the size of the orifice on the air inlet tube, air flow is controlled. This is inexpensive but not a very accurate method of controlling inhalation. A user typically has different capacity or ability to inhale which varies as to the type of user as well as the physical condition of the user. These differences result in a change of air flow. Restricting the air inlet tube too much limits the ability of the medication to be delivered at a desired or necessary rate. It also is typically necessary to train the user with the device to obtain the desired result. Such training may be difficult again because of the physical condition of the user, for example if the user is an infant or a non-human animal.

Thus, there is a need for an inhalation device that will expel a dose of medication into the gas stream at a pre-selected speed of the gas. There is a further need for an inhalation device that will dispense medication only one direction of gas flow through the inhalation device.

### SUMMARY OF THE INVENTION

There is provided an inhalation device to deliver a pre-selected dose of medication to a user. The inhalation device comprises an enclosure having an inhalation tube. The inhalation tube has an inlet end and an outlet end. A medication dispenser is coupled to the enclosure, with at least a portion of the dispenser extending into the inhalation tube. A sensor is mounted in the enclosure with at least a portion of the sensor extending into the inhalation tube. The sensor has a characteristic of bending in proportion to speed of gas flowing in a given direction within the inhalation tube. An electrical circuit is coupled to the sensor and medication dispenser. The electrical circuit is configured to trigger the medication dispenser upon receipt of a signal from the sensor at a predetermined gas flow speed in the inhalation tube. The electric circuit provides an output signal wherein a dose of medication is expelled into the inhalation tube. Another embodiment provides a medication reservoir coupled to the medication dispenser.

There is also provided an inhalation device to deliver a pre-selected dose of medication to a user. The inhalation device comprises an enclosure having an inhalation tube. The inhalation tube has an inlet end and an outlet end. A medication dispenser is coupled to the enclosure, with at least a portion of the dispenser extending into the inhalation tube. An electrical circuit is coupled to the sensor and medication dispenser. A thermal compensater is coupled in the enclosure with at least a portion extending into the inhalation tube and coupled to the electrical circuit. A thermal compensater senses the temperature and humidity of gas flowing in the inhalation tube. The electrical circuit is configured to trigger the medication dispenser upon receipt of a signal from the sensor that a predetermined gas flow speed has been reached in the inhalation tube, wherein a dose of medication is expelled into the inhalation tube.

There is also provided a method of medication delivery. The method comprises the steps of providing an inhalation device having an inhalation tube. Mounting a sensor in the inhalation device with at least a portion of the sensor extending into the inhalation tube. The sensor having a characteristic of bending in proportion to speed of gas flowing in a given direction within the inhalation tube. Mounting a medication dispenser in the inhalation device, with at least a portion of the dispenser extending into the inhalation device. Mounting an electrical circuit in the inhalation device and coupling the circuit to the sensor and medication dispenser. Configuring the electrical circuit to trigger the medication dispenser upon receipt of a signal from the sensor at a predetermined gas flow speed in the inhalation tube. Expelling a dose of medication into the inhalation tube in response to the signal. Another embodiment includes the step of mounting a thermal compensator in the inhalation device with at least a portion extending into the inhalation tube and coupling the thermal compensator to the electrical circuit to control temperature and humidity of gas flowing in the inhalation tube.

### BRIEF DESCRIPTION OF THE DRAWINGS

FIG. 1 is a schematic illustration of an exemplary embodiment of an inhalation device including a sensor mounted with at least a portion extending into an inhalation tube, the sensor having a characteristic of bending in proportion to speed of gas flowing in a given direction within the inhalation tube.

FIG. 2 is a schematic of an exemplary embodiment of an electrical circuit mounted in an inhalation device, the circuit conditions a signal from a sensor responsive to speed of gas flowing in the inhalation device.

### DETAILED DESCRIPTION OF THE EXEMPLARY EMBODIMENTS

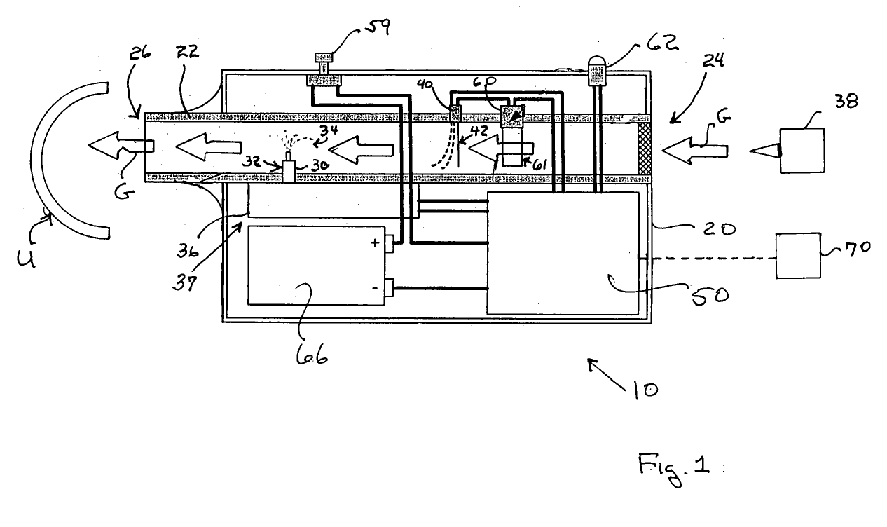

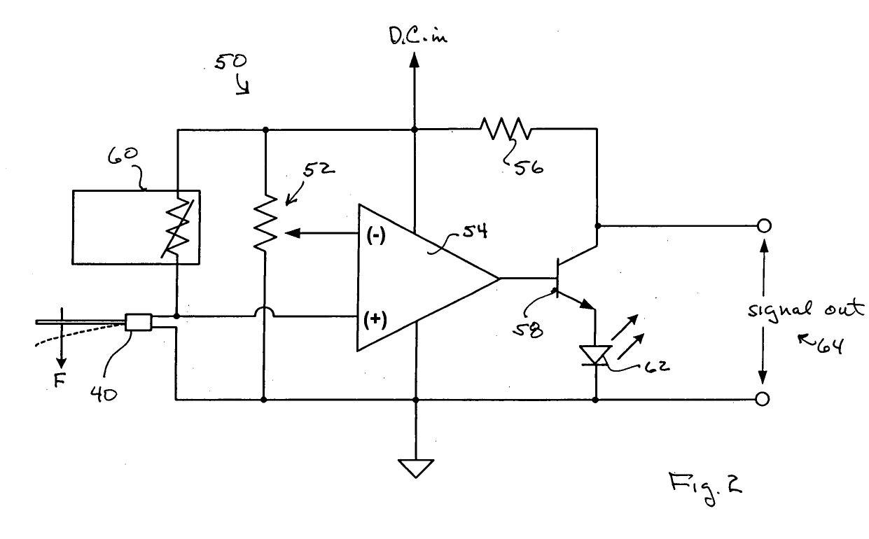

Referring now to FIGS. 1 and 2, there is illustrated an exemplary embodiment of an inhalation device that provides a passageway for the transport of inspired gas, with a sensor in the passageway, to detect the gas flow in the passageway and trigger the release of a dose of medication, in powdered or micronized particulate medication dispensed by Electro-Hydro Dynamic dispersal (EHD) or fluid (i.e. saline solution with medication) into the gas flow and ultimately into the lungs of the inspiring user. For purposes of this application, it should be understood that the term “gas” includes what is conventionally referred to as “air” and is breathable by most animals such as humans, dogs, cats, and the like. However, it should also be understood that gas can be any combination of gaseous elements delivered to a user by manual or mechanical means such as an oxygen pump, inhaler, air compressor, ventilator, respirator, aqua lung, or the like.

For purposes of this application, it should be understood that a user U is typically an animal such as a human being, a dog, horse, or the like. Also, the user U may be assisted by a healthcare giver, for example a nurse, physician, spouse, etc.

Referring now to FIG. 1, there is illustrated an inhalation device 10 to deliver a preselected dose of medication 34 to a user U. The inhalation device 10 comprises an enclosure 20 having an inhalation tube 22. The inhalation tube 22 has an inlet end 24 and an outlet end 26. The enclosure 20 and the inhalation tube 22 can be composed of any suitable and convenient material such as plastic or metal with sufficient strength and durability to accommodate the type of medication and use anticipated of the device. A filter or screen can be installed at the inlet end 24 of the inhalation tube 22 as is determined by the manufacturer or user U of the inhalation device 10.

A medication dispenser 30 is coupled to the enclosure 20 with at least a portion 32 of the dispenser 30 extended into the inhalation tube 22. The medication dispenser 30 can operate by any convenient and conventional manner such as a pump, or an electrical discharge, or an Electro-Hydro Dynamic dispersal or a solenoid.

A sensor 40 is mounted in the enclosure 20 with at least a portion 42 extending into the inhalation tube 22. The sensor 40 has a characteristic of bending in proportion to speed of gas G flowing in a given direction within the inhalation tube 22. In FIG. 1, the gas flow is shown from the right to left (large open arrows) and the portion 42 of the sensor 40 is shown moving to the left or towards the user U. When gas G is inhaled by the user U, the gas G passes through the inhalation tube 22 and will apply a force F on the sensor 40. The sensor 40 will then bend or flex. Such bending or flexing of the sensor 40 will create a change in resistance in the sensor 40. Such change in resistance is detected by the electrical circuit 50, which will be described below. The sensor 40 can be a deflectable substrate having a conductive ink deposited thereon in a pattern to form a flexible potentiometer in which the resistance is consistently and predictably changed upon deflection or bending of the substrate in one direction. Examples of such bending or flex sensor is disclosed in U.S. Pat. Nos. 5,086,785, 5,157,372, and 5,309,135.

The inhalation device 10 also includes an electrical circuit 50 coupled to the sensor 40 and the medication dispenser 30 with the electrical circuit 50 configured to trigger the medication dispenser 30 with a signal 64 upon receipt of a signal from the sensor 40 at a predetermined gas G flow speed in the inhalation tube 22. A dose of medication 34 is then expelled by the medication dispenser 30 into the inhalation tube 22 and is inspired by the user U.

FIG. 2 is an exemplary embodiment of an electrical circuit 50 for use in an inhalation device 10. Gas flowing through the inhalation tube 22 creates a force F on the sensor 40 as depicted in FIG. 2. The deflection of the portion 42 of the sensor extending into the inhalation tube 22 changes the resistance of the sensor 40 which in turn changes the voltage across the sensor 40. The voltage across the sensor 40 is detected by the operational amp 54 which is configured as a comparator.

A reference voltage is set by potentiometer 52. The reference voltage on the potentiometer 52 is the trigger point at which the dose of medication 34 is to be released based on the analog output from the sensor 40. By changing the reference voltage with the potentiometer 52, it calibrates the inhalation device 10 to disperse a dose of medication 34 at a given speed of gas G flowing through the inhalation tube 22. When the speed of the gas G in the inhalation tube 22 reaches the pre-selected or appropriate speed, the trigger signal is sent to expel the dose of medication 34.

In the electrical circuit 50 illustrated in FIG. 2, a comparator circuit is used to trigger a dose of medication 34. To calibrate the electrical circuit 50, a known constant gas flow rate is applied in the inhalation tube 22, the electrical circuit 50 is powered and, a trigger point (dispensing of the medication) is set. At such desired flow rate, the operator adjusts the potentiometer 52 until the trigger light 62 or other suitable indicator (for example, an audible signal or other visual or tactile signal) is activated. The trigger point is now set. It should be understood that one or more resistors of suitable size and power rating can be used instead of the potentiometer 52 to set the trigger point.

Another embodiment will couple a micro controller having an analog/digital (A/D) capability, to the electrical circuit 50. The electrical circuit 50 is configured to output an analog voltage proportional to the gas G flow in the inhalation tube 22. The analog voltage would be input to the A/D of the micro controller to trigger the medication at a pre-determined gas flow rate.

Referring again to FIG. 2, the voltage from the sensor 40 is an input into the plus (+) side of the comparator 54 and is compared with the voltage from the variable resistor 52 in the minus (−) side of the comparator 54. When the voltage from the sensor 40 rises above or falls below the reference voltage of the potentiometer 52, the comparator 54 changes states. The output from the comparator 54 drives the base of the transistor 58 thereby turning the transistor 58 on. An output signal 64 is then generated and triggers the medication dispenser 30 to disperse a dose 34 of medication into the inhalation tube 22. The circuit can also include a trigger light 62 which indicates that a dose of medication 34 has been expelled into the inhalation tube 22. The trigger light 62 can be, for example a light emitting diode of any suitable color or other type of suitable visual indicator.

The electrical circuit 50 can also include a thermal compensation circuit 60 placed in series with the sensor 40. The thermal compensator 60 is mounted in the enclosure 20 with at least a portion 61 extending into the inhalation tube 22 and coupled to the electrical circuit 50 to sense temperature and humidity of gas G flowing in the inhalation tube 22. Environmental conditions in which the inhalation device 10 is placed, such as temperature and humidity, can affect the set trigger point at which a dose of medication 34 is dispensed into the inhalation tube 22. If the environmental conditions are of sufficient magnitude, it may be necessary to compensate for such conditions. In the exemplary embodiment of the electrical circuit 50 illustrated in FIG. 2, a thermal compensation circuit 60 having similar properties to the sensor 40 is used. The sensor 40 and thermal compensation circuit 60 are configured in series. The thermal compensator circuit 60 senses the same environment conditions affecting the sensor 40 and makes appropriate adjustments, for example, a voltage adjustment. Such configuration electrically cancels out the environmental conditions to which the sensor 40 is subjected in a given situation. The effect of each configuration is to maintain the set trigger point for a dose of medication 34 as described above. The thermal compensator 60 can be positioned between the inlet end 24 of the inhalation tube 22 and the sensor 40. Electrical circuit 50 can also include current limiting resistors such as 56 to properly condition the signal 64 to be generated by the electrical circuit 50.

It should be understood that other ways of adjusting for environmental conditions can be utilized. For example, a micro controller can be used, having a look-up table or an executable mathematical equation, to correct for environmental effects on the gas flow in the inhalation tube 22.

One embodiment of the inhalation device 10 includes a direct current power source 66 which is coupled to the electrical circuit 50, the sensor 40 and the medication dispenser 30. The typical direct current power source is a 9-volt battery, however it should be understood that any suitable battery configuration and voltage can be used or an external power source with a properly configured output voltage can be connected to the inhalation device 10 to provide sufficient power to the inhalation device 10 components. The electrical circuit can be mounted in the enclosure 20 or can be mounted external to the inhalation device 10 but electrically connected to the various components within the inhalation device 10.

Inhalation device 10 can also include a medication reservoir 36 coupled to the medication dispenser 30. The medication reservoir 36 can be any suitable device, for example, a blister pack, capsule, tube, or the like. The medication reservoir 36 may be disposable or refillable. The medication reservoir 36 can be mounted in the inhalation device 10 or it can be external to the inhalation device 10 and fluidly in communication with the medication dispenser 30. The medication dispenser 30 is positioned between the outlet end 26 of the inhalation tube 22 and the sensor 40. It should be understood that the medical reservoir 37 can be a single use or a refillable medication reservoir.

The movement of gas G through the inhalation tube 22 can be facilitated by the intake of breath by the user U, also referred to as manual operation. The gas G flow can also be facilitated by a means for pumping gas 38 such as provided for example by a respirator.

One embodiment of inhalation device 10 is configured to be hand-held with one hand of the user U and is of such size and weight to be easily lifted to the mouth of the user U administering the inhalation therapy. In most instances, the inhalation device 10 can be used for the self-administration of medication by inhalation by the user U.

Another embodiment of the inhalation device 10 includes a communication module 70. The communication module 70 is coupled to the electrical circuit 50. The communication module 70 can be mounted in the enclosure 20 of the inhalation device 10 or it can be external to the inhalation device 10. The communication module contains circuitry for storing, cataloging, transmitting and receiving information and instructions relating to the inhalation device 10. For example, the communication device can be hardwired to a computer microprocessor or it can include wireless transmission and receiving circuitry, such as Bluetooth or WIFI technology. The communication module 70 can be used by the user U or by a health provider assisting the user U to change the parameters of the inhalation device 10 such as the amount or frequency of the dose 34 of medication. The communication module 70 can also store data such as rate of flow of the gas G through the inhalation tube 22, the peak flow rate of the gas G, the data and time of day when a dose 34 of medication was expelled into the inhalation device 22. The communication module 70 can also be used to change any of such parameters or such other information and instructions as determined by the user U or an assisting health provider. It is also contemplated that the communication module 70 can be integrated with the electrical circuit 50 on a single circuit board.

A method of medication delivery comprises the steps of providing an inhalation device 10 having an inhalation tube 22. Then mounting a sensor 40 in the inhalation device 10 with at least a portion 42 of the sensor 40 extending into the inhalation tube 22. The sensor 40 has a characteristic of bending in portion to speed of gas G flowing in a given direction within the inhalation tube 22. Mounting a medication dispenser 30 in the inhalation device 10 with at least a portion 32 of the dispenser 30 extending into the inhalation device 10. Mounting an electrical circuit 50 in the inhalation device 10 and coupling the circuit 50 to the sensor 40 and the medication dispenser 30. Configuring the electrical circuit 50 to trigger the medication dispenser 30 upon receipt of a signal 64 from the sensor 40 at a predetermined gas G flow speed in the inhalation tube 22 and expelling a dose 34 of the medication into the inhalation tube 22 in response to the signal 64.

The method can also include the steps of mounting a thermal compensator 60 in the inhalation device 10 with at least a portion 61 of the thermal compensator 60 extending into the inhalation tube 22 and coupling the thermal compensator 60 to the electrical circuit 50 to control temperature and humidity of gas G flowing in the inhalation tube 22. A medical reservoir 37 can be coupled to the medication dispenser 30 to supply a fixed or refillable amount of medication to the user U. The method can also include the steps of providing a communication module 70 coupled to the electrical circuit 50 and using the communication module 70 to catalog, transmit, store and receive data and instructions. The method can also include the step of calibrating the electrical circuit 50 to trigger the medication at the predetermined gas flow speed as described above.

It should be noted that the sensor 40 will produce a change in voltage in response to gas flow in only one direction. For example, if a user U were to cough or inadvertently blow into the outlet end 26 of the inhalation tube 22, the sensor 40 would not respond and therefore no change in voltage would be detected by the comparator 54 and no output signal 64 generated to trigger a dose 34 of medication.

Although the disclosed embodiments have been described in detail, it should be understood that various changes, substitutions and alterations can be made to the embodiments without departing from their spirit and scope.

What is claimed is:

1. An inhalation device to deliver a pre-selected dose of medication to a user, the inhalation device comprising:
an enclosure having an inhalation tube, the inhalation tube having an inlet end and an outlet end;
a medication dispenser coupled to the enclosure, with the dispenser in communication with the inhalation tube;
a sensor mounted in the enclosure with at least a portion extending into the inhalation tube, the sensor having a characteristic of bending in proportion to speed of gas flowing in a given direction within the inhalation tube; and
an electrical circuit coupled to the sensor and medication dispenser, with the electrical circuit configured to trigger the medication dispenser, upon receipt of a signal from the sensor at a predetermined gas flow speed in the inhalation tube, wherein a dose of medication is expelled into the inhalation tube.

2. The inhalation device of claim 1, including a medication reservoir coupled to the medication dispenser.

3. The inhalation device of claim 1, including a thermal compensator mounted in the enclosure with at least a portion extending into the inhalation tube and coupled to the electrical circuit to sense temperature and humidity of gas flowing in the inhalation tube.

4. The inhalation device of claim 3, wherein the thermal compensator is positioned between the input end of the inhalation tube and the sensor.

5. The inhalation device of claim 1, wherein the medication dispenser is positioned between the output end of the inhalation tube and the sensor.

6. The inhalation device of claim 1, including one of a disposable medication reservoir and a refillable medication reservoir mounted in the enclosure and fluidly coupled to the medication dispenser.

7. The inhalation device of claim 1, wherein the electrical circuit is mounted in the enclosure.

8. The inhalation device of claim 1, wherein the medication is one of a powder and a liquid.

9. The inhalation device of claim 1, including an indicator coupled to the electrical circuit to indicate that medication has been delivered.

10. The inhalation device of claim 1, wherein the sensor responds only during an intake of gas by the user of the inhalation device.

11. The inhalation device of claim 10, wherein the gas is moved through the inhalation tube by a means for pumping gas.

12. The inhalation device of claim 1, wherein the sensor is a variable resistor.

13. The inhalation device of claim 1, wherein the electrical circuit, the sensor and the medication dispenser are coupled to a direct current power source.

14. The inhalation device of claim 1, wherein the user is a human being.

15. The inhalation device of claim 1, including a means for calibrating the electrical circuit.

16. The inhalation device of claim 1, including a communication module coupled to the electrical circuit for cataloging, transmitting, store and receiving data and instructions.

17. An inhalation device to deliver a pre-selected dose of medication to a user, the inhalation device comprising:
an enclosure having an inhalation tube, the inhalation tube having an inlet end and an outlet end;
a medication dispenser coupled to the enclosure and in communication with the inhalation tube;
a sensor mounted in the enclosure with at least a portion extending into the inhalation tube, the sensor having a characteristic of bending in proportion to speed of gas flowing in a given direction within the inhalation tube;
an electrical circuit coupled to the sensor and medication dispenser; and
a thermal compensator mounted in the enclosure with at least a portion extending into the inhalation tube and coupled to the electrical circuit to sense temperature and humidity of gas flowing in the inhalation tube, with the electrical circuit configured to trigger the medication dispenser upon receipt of a signal from the sensor at a predetermined gas flow speed has been reached in the inhalation tube, wherein a dose of medication is expelled into the inhalation tube.

18. The inhalation device of claim 17, including a medication reservoir coupled to the medication dispenser.

19. The inhalation device of claim 17, wherein the thermal compensator is positioned between the input end of the inhalation tube and the sensor.

20. The inhalation device of claim 17, wherein the medication dispenser is positioned between the output end of the inhalation tube and the sensor.

21. The inhalation device of claim 17, including one of a disposable medication reservoir and a refillable medication reservoir mounted in the enclosure and fluidly coupled to the medication dispenser.

22. The inhalation device of claim 17, wherein the electrical circuit is mounted in the enclosure.

23. The inhalation device of claim 17, wherein the medication is one of a powder and a liquid.

24. The inhalation device of claim 17, including an indicator coupled to the electrical circuit to indicate that medication has been delivered.

25. The inhalation device of claim 17, wherein the sensor responds only during an intake of gas by the user of the inhalation device.

26. The inhalation device of claim 25, wherein the gas is moved through the inhalation tube by a means for pumping gas.

27. The inhalation device of claim 17, wherein the sensor is a variable resistor.

28. The inhalation device of claim 17, wherein the electrical circuit, the sensor and the medication dispenser are coupled to a direct current power source.

29. The inhalation device of claim 17, wherein the user is a human being.

30. The inhalation device of claim 17, including a means for calibrating the electrical circuit.

31. The inhalation device of claim 17, including a communication module coupled to the electrical circuit for cataloging, transmitting, store and receiving data and instructions.

32. A method of medication delivery, the method comprising the steps of:
providing an inhalation device having an inhalation tube;
mounting a sensor in the inhalation device with at least a portion of the sensor extending into the inhalation tube, the sensor having a characteristic of bending in proportion to speed of gas flowing in a given direction within the inhalation tube;
mounting a medication dispenser in the inhalation device, the dispenser in communication with the inhalation tube;
mounting an electrical circuit in the inhalation device and coupling the circuit to the sensor and medication dispenser;
configuring the electrical circuit to trigger the medication dispenser upon receipt of a signal from the sensor at a predetermined gas flow speed in the inhalation tube; and
expelling a dose of the medication into the inhalation tube in response to the signal.

33. The method of claim 32, including the steps of mounting a thermal compensator in the inhalation device with at least a portion extending into the inhalation tube and coupling the thermal compensator to the electrical circuit to sense temperature and humidity of gas flowing in the inhalation tube.

34. The method of claim 32 including the step of coupling a medication reservoir to the medication dispenser.

35. The method of claim 32 including the steps of providing a communication module coupled to the electrical circuit and using the communication module to catalog, transmit, store and receive data and instructions.

36. The method of claim 32, including the step of calibrating the electric circuit to trigger the medication at the predetermined gas flow speed.
